(19) United States
(12) Patent Application Publication
(10) Pub. No.: US 2022/0126016 A1
(43) Pub. Date: Apr. 28, 2022

(54) UNIVERSAL CLAMPING AND CONNECTION SYSTEM

(71) Applicant: Pryor Products, Inc., Oceanside, CA (US)

(72) Inventors: James Mark COX, Oceanside, CA (US); Richard Enoch QUINTANIA, Oceanside, CA (US); Kevin DAILEY, Oceanside, CA (US); Kevin DONAHUE, Oceanside, CA (US); George KEMPER, Oceanside, CA (US)

(73) Assignee: Pryor Products, Inc., Oceanside, CA (US)

(21) Appl. No.: 17/508,938

(22) Filed: Oct. 22, 2021

Related U.S. Application Data
(60) Provisional application No. 63/104,482, filed on Oct. 22, 2020.

(51) Int. Cl. A61M 5/14; F16M 13/02

(52) U.S. Cl. CPC A61M 5/1415; F16M 2200/063; F16M 13/02

(57) ABSTRACT

A clamping and connection system configured to be pivotally attached to two pieces of equipment such as an IV pole and a hospital bed. The clamping and connection system includes a clamping portion and an attachment portion. The system is configured to be rotatable around the attachment portion to allow optimal placement of the pole while in transport and connected to the bed.

### CROSS-REFERENCE TO RELATED APPLICATION

This application claims the benefit of and priority to U.S. Provisional Patent Application No. 63/104,482, filed Oct. 22, 2020. The foregoing provisional patent application is incorporated by reference herein in its entirety.

### GENERAL DESCRIPTION

In certain applications, devices and equipment must be mounted from a support in proximity to the location where the equipment is utilized. This requirement is especially common with portable medical equipment that must be supported near where the equipment is utilized for treatment of patients.

Clamps are frequently used to mount equipment and devices to existing supports such as tubular supports as in intravenous poles aka IV poles (vertical) and bed rails (horizontal). Clamps have also been utilized with planar supports such as table edges.

A typical clamp may be a c-shaped clamp that grasps a support between a fixed jaw and a moveable jaw. The jaw operator mechanism for moving the moveable jaw into engagement with and securely grasping the support has typically utilized a screw carried in a threaded opening in the clamp body. This screw, of necessity, must be of a length greater than the maximum distance between the fixed and moveable jaws plus the width of the clamp body through which it is threaded. This creates an elongated profile that may interfere with other clamps or structures carried on the support. Another feature which is desirable in equipment clamps is the ability to rotate attached equipment so that the equipment will be presented to the user in an upright orientation. In the past, in order to allow for the clamp to have rotational capability, projections from the clamp body have been required in order to accommodate the rotational mechanism which further increases the profile of the clamp and the potential for interference with other clamps or structures on the support.

It is also desirable for the clamp to enable easier movement of the attached equipment. By allowing more degrees of freedom, the equipment may be more flexible and adaptable to the environment. This would allow the clamped equipment to move more freely during movement or transportation of the attached or linked equipment.

Clamps are used to support equipment of various weights. There is a need for versatile clamps that can support heavy medical equipment. These clamps should provide secure and stable mounting and be easy and convenient to use.

The present application discloses various embodiments of clamp system that include mechanisms that provide for securely mounting the clamp to a support and minimize the profile the clamp while providing versatility in view of the ever-expanding size and weight of equipment being mounted. The disclosed clamps are not solely for equipment support. The disclosed clamps may be used for clamping structural elements together. Also, a link arm may be attached to the end of the clamp so that the clamp and the link arm can provide a system for connecting to devices together while being spaced by the link arm. For example, an IV pole may connected to a bed or bed frame via a clamp(s) and link arm. The system and arrangement may allow the pole and bed to be easily moved together without having to disconnect the IV pole from the bed or require two or more people to move the pole and bed together.

### BRIEF DESCRIPTION OF THE DRAWINGS

The features, aspects, and advantages of the disclosed deliver system will become apparent from the following description, and the accompanying exemplary embodiments shown in the drawings, which are briefly described below.

FIG. 1 shows an exemplary clamping and connection system from a front side view.

FIG. 2 shows an exemplary clamping and connection system shown in FIG. 1 from a rear side view.

FIG. 3 shows an exemplary clamping and connection system shown in FIG. 1 from a side view.

FIG. 4 shows an exemplary clamping system and connection shown in FIG. 1 from a top view.

FIG. 5A shows a close up of the clamping portion of the exemplary clamping and connection system shown in FIG. 1.

FIG. 5B shows sectional view A-A shown in FIG. 5A.

FIG. 6A shows a close up of the clamping portion of the exemplary clamping and connection system shown in FIG. 1.

FIG. 6B shows sectional view B-B shown in FIG. 6A.

FIG. 7A shows a top view of the exemplary clamping and connection system shown in FIG. 1.

FIG. 7B shows sectional view C-C shown in FIG. 7A.

FIG. 8 shows an exemplary embodiment of a clamping and connection system utilized with a hospital bed.

FIG. 9 shows a top view of possible adjustments of the exemplary clamping and connection system.

FIG. 10 shows another exemplary embodiment of the clamping and connection system.

FIG. 11 shows an exemplary embodiment of the clamping and connection system when an exemplary attachment pin is engaged.

FIG. 12 shows the exemplary embodiment of the clamping and connection system in FIG. 11 when the exemplary attachment pin is disengaged.

FIG. 13 shows another exemplary embodiment of the clamping and connection system.

FIG. 14A shows the top view of the clamping and connection system of FIG. 13.

FIG. 14B shows the side view of the clamping and connection system of FIG. 13.

FIG. 15 shows an exemplary method of assembling the clamping and connection system of FIG. 13.

### DETAILED DESCRIPTION

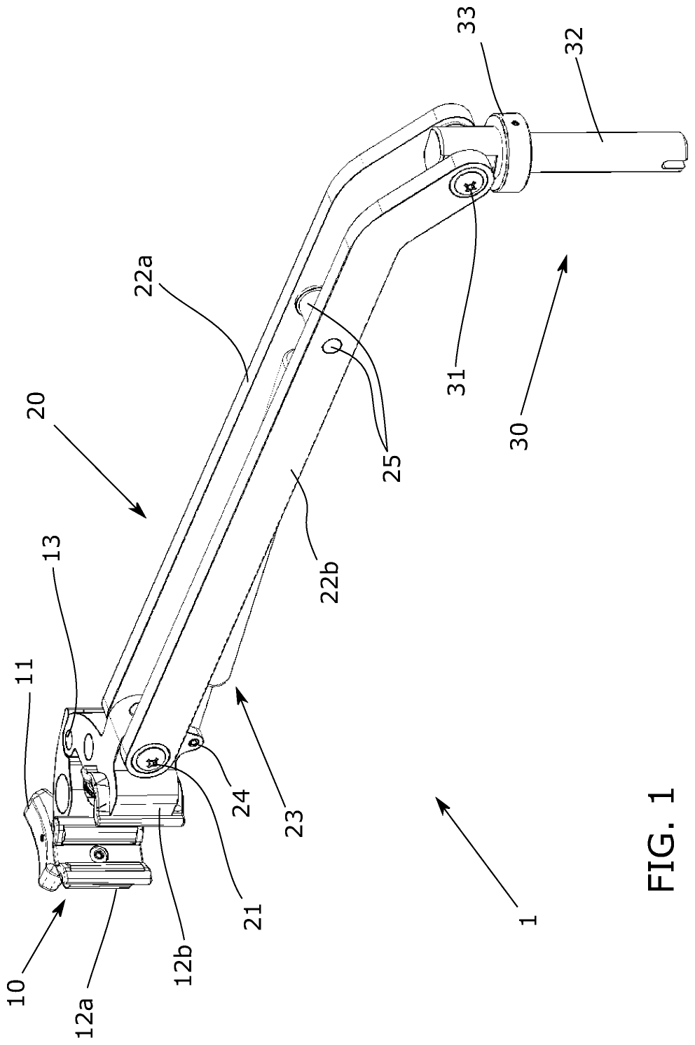

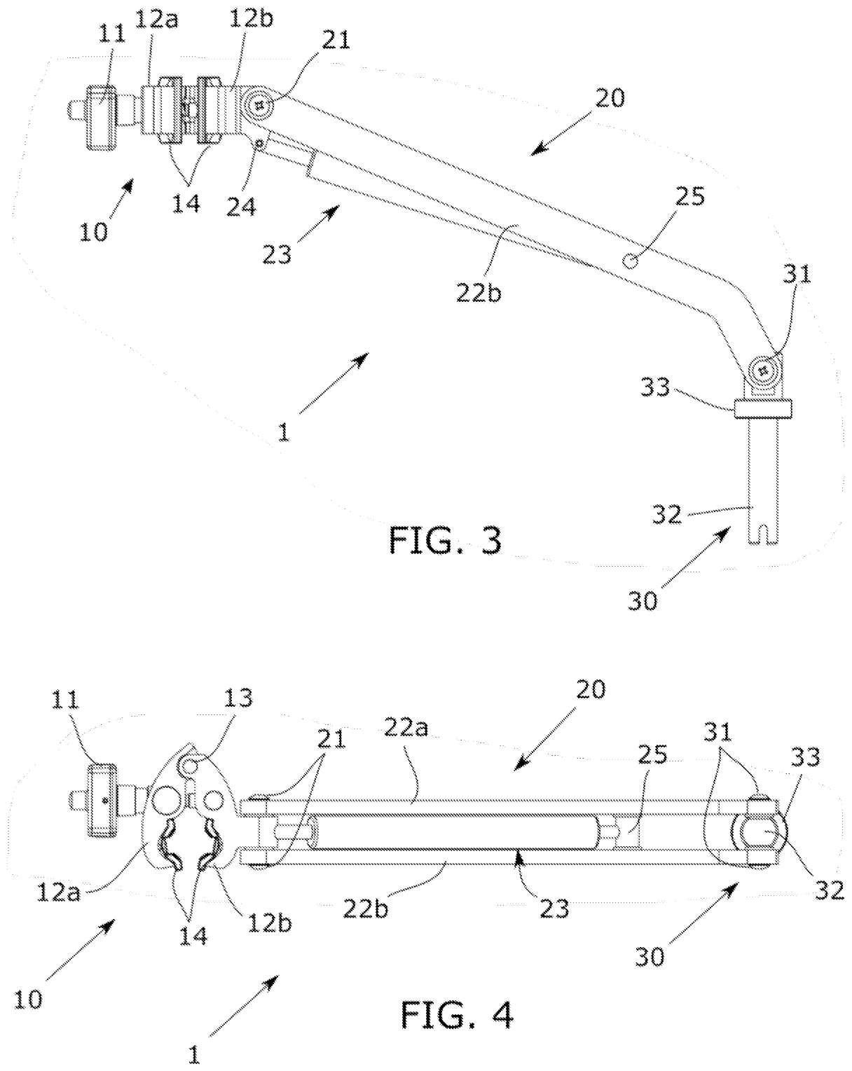

FIGS. 1-4 illustrates an exemplary clamping and connection system 1 having a clamp portion 10, a link arm portion 20, and attachment portion 30. The clamp portion 10 is configured to clamp to various equipment and/or medical devices (e.g. a movable pole for medication equipment such as IV's and IV pumps or tables) to a support. Such support may include other medical devices or equipment such as beds, wheel chairs, or walkers. The clamp utilized may also include the mechanism disclosed in U.S. Pat. No. 10,738,808 which is incorporated by reference herein. Such a mechanism allows the clamp to resist loosening while still providing for manual operation.

The clamp portion 10 includes a knob 11 configured to open and close a first clamp arm 12a and second clamp arm 12b. Turning the knob 11 allows the clamp portion 10 to tighten or loosen (i.e. clamped or unclamped). The clamp portion includes a clamp pivot 13 to which the first and second clamp arm 12a/12b are attached. The clamp pivot 13 may be formed as a pin, bar or rod in a generally cylindrical shape to permit relative rotation or pivoting of the connected arms. The clamp pivot 13 allows the first clamp arm 12a to rotate and move relative to second clamp arm 12b in order for the clamp 10 to be tightened or loosened. The first and second clamp arms 12a/12b may include one or more friction pads 14. The friction pads 14 may be formed from a rubber or plastic with a higher coefficient of friction than the surface of the first and second clamp arms 12a/12b. The friction pads 14 may be fastened to the first and second clamp arms 12a/12b. Alternatively, a larger area of the clamp arms 12a/12b may be covered (e.g., over molded) with a suitable non-slip surface. In yet another embodiment, the clamp arms 12a/12b may be entirely coated or entirely covered with a non-slip surface. The clamp arms 12a/12b and friction pads may be formed in a V, U shape, or any other shape that may accommodate a rod or pole shaped feature to be clamped by the clamp system 1.

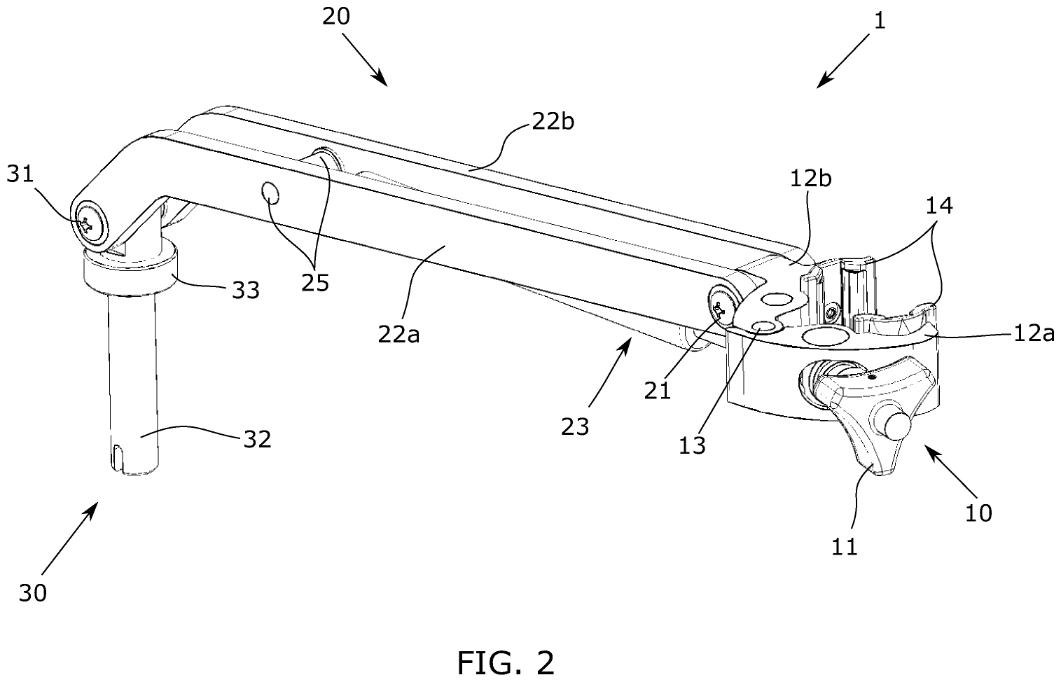

The second clamp arm 12b is attached to the link arm portion 20 via a link arm pivot 21. The pivot 21 may be formed as a pin, bar or rod in a generally cylindrical shape to permit relative rotation or pivoting of the connected clamp 10 and link arm portion 20. Link arm portion 20 is configured to be rotatable relative to the clamp portion 10 via a link arm pivot 21. The link arm portion 20 may include two or more arms or members. As shown in the embodiment disclosed in FIGS. 1 and 2, a first link member 22a and a second link member 22b may be provided. The link members may be configured to be parallel to each other and are attached to the second clamp arm 12b at the link arm pivot 21. The link arm pivot 21 may be located at a first end of the link arm portion 20. The link arm portion 20 may also include a dampener 23 configured to be attached to the second clamp arm 12b at a first dampener pivot 24 and attached to the two link members 22a, 22b at a second dampener pivot 25. The dampener 23 may be positioned between the first link member 22a and second link member 22b and may be configured to be parallel to both link members 22a/22b. As shown in FIG. 1, the link arm 20 may include a bend or curve. Alternatively, the link arm 20 could be formed in a relatively straight configuration. Each pivot 21, 24, 25, 31 may include an axis of rotation that is parallel to the other pivots and also perpendicular to the axis of the equipment or pole 100. The axis of rotation for each of the pivots 21, 24, 25, 31 may also be perpendicular to the longitudinal axis of attachment pin 32.

The attachment portion 30 may be connected to the link members 22a/22b at an attachment pivot 31. The attachment pivot 31 may be located at a second end of the link arm 20 which is at the opposite of the first end of the link arm portion 20 where the link arm pivot 21 is located. The pivot 31 may be formed as a pin, bar or rod in a generally cylindrical shape to permit relative rotation or pivoting of the connected link arm portion 20 and attachment portion 30. The attachment portion 30 is configured to be rotatably attached to the first and second link members 22a/22b. The attachment portion includes an attachment pin 32 attached to the link members 22a/22b at the attachment pivot. The attachment pin 32 is configured to be supported to a receiving end (not shown) of the attached supporting equipment or medical device. A bumper 33 is attached and surrounds a portion of the attachment pin 32 proximate the attachment pivot 31. The bumper 33 is configured to prevent the pin 32 from being inserted too far into the supporting equipment (e.g., bed frame) or medical device. The bumper 33 may act as a support for the attachment pin 32 when the pin 32 is connected to the receiving end of the supporting equipment or medical device.

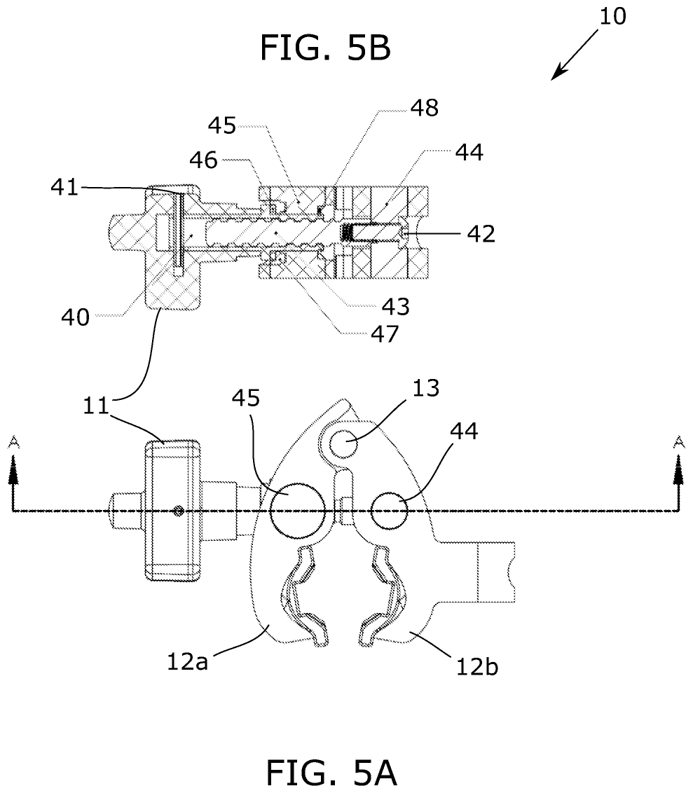

FIG. 5A and 5B illustrate a close up and a sectional view, respectively, of the clamp portion 10 along line “A-A”. As shown in FIG. 5B, a thrust nut 40 is embedded in the knob 11. The thrust nut 40 is rotationally locked relative to the knob 11 via a thrust nut pin 41. A speed screw 43 is fixed onto a screw pivot axle 44 by a bolt 42 located on the second clamp arm 12b. The second clamp arm 12b may be rotatable relative to the screw pivot axle 44. The speed screw 43 is configured to be inserted into the thrust nut 40 such that the rotation of the knob 11 and thrust nut 40 allows the clamp arms 12a and 12b to move relative to each other in order to clamp or unclamp. As the knob is rotated in the tightening direction, the thrust nut 40 will move closer towards the end of the speed screw 43 and closer to the speed screw pivot axle 44. A thrust nut pivot axle 45 is configured to be embedded in the first clamp arm 12a. The thrust nut pivot axle 45 is configured to translate the movement of the thrust nut 40 onto the first clamp arm 12a. The thrust nut 40 is configured to abut the thrust nut pivot axle 45 via a friction washer 46 and friction washer housing 47. The first clamp arm 12a may be rotatable relative to the thrust nut pivot axle 45 in order to provide the clamping mechanism of the clamping portion 10. Similarly, rotating the knob in a loosening direction allows the clamping arms 12a and 12b to unclamp or open. A retaining ring 48 is located on the opposite side of the thrust nut pivot axle relative to the friction washer housing 47. Retaining ring 48 is configured to affix the thrust nut 40 to the thrust nut pivot axle 45, especially during the loosening action.

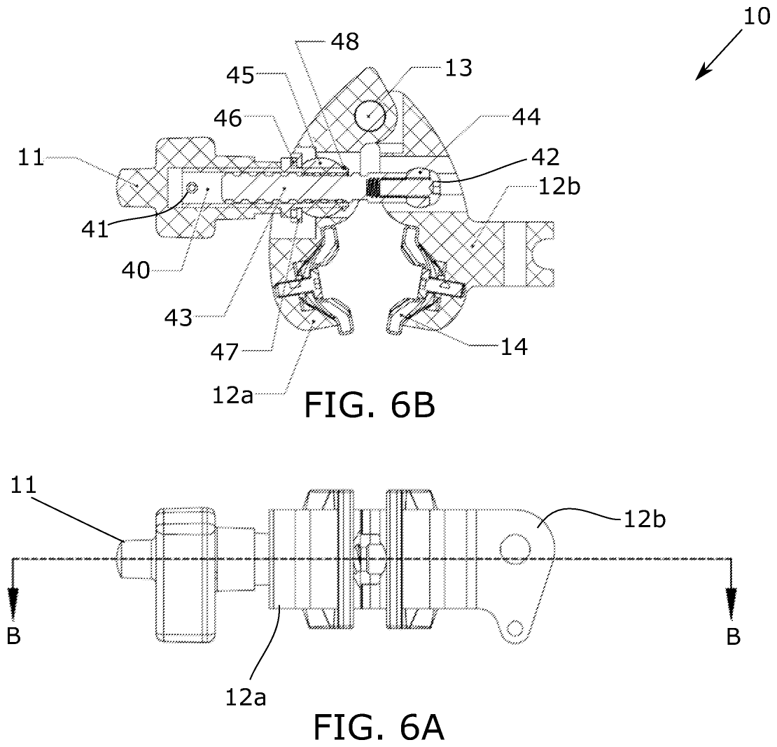

FIG. 6A-6B illustrate an alternative close cup and sectional view, respectively, of the clamp portion 10 along line “B-B”. As shown, the second clamp arm 12b may include a first pivot opening 50 for the link arm pivot 21 and a second pivot opening 51 for dampener pivot 24.

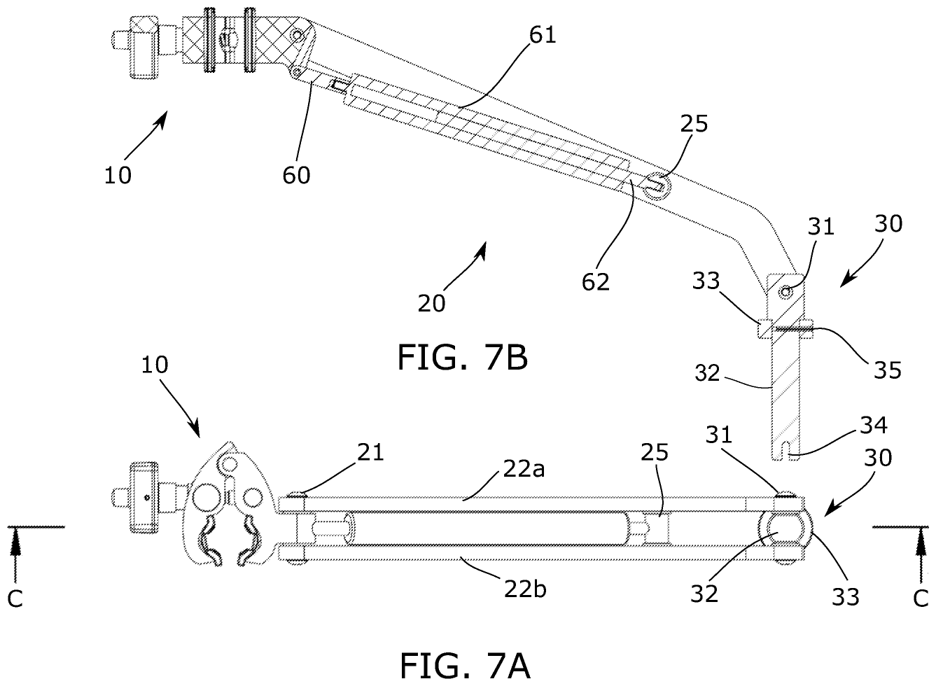

FIG. 7B illustrates a longitudinal sectional view of the clamp system 1 along line C-C. The dampener 23 is configured to smooth movement and rotation of the link arm portion 20. An exemplary simplified schematic of the dampener 23 is shown in FIG. 7A. The dampener 23 may be hydraulic or pneumatic. The dampener 23 may include a dampener body 61 attached to a dampener end 60. The dampener end 60 is pivotably attached to the second link arm 23b via pivot 21. A dampener rod 62 is housed at least partially within the dampener body 61 and is configured to translate longitudinally relative to the dampener body 61. The dampener rod is pivotably attached to first and second link members 22a/22b via pivot 25. The pivot 25 may be formed as a pin, bar or rod in a generally cylindrical shape to permit relative rotation or pivoting of the connected dampener 23 and link arm portion 20.

The attachment portion 30 includes the attachment pin 32 affixed to the attachment pivot 31. The attachment pin 32 may include a cutout 34 which is configured to attach to a receiving part of the support (e.g. bed, table, wheel chairs, walkers, etc.). The cutout 34 may include different shapes and features in order to affix to the receiving part of the support. Bumper 33 may be locked to the attachment pin 32 via the bumper pin 35. The bumper 33 allows the attachment pin to sit properly in the receiving portion of the support

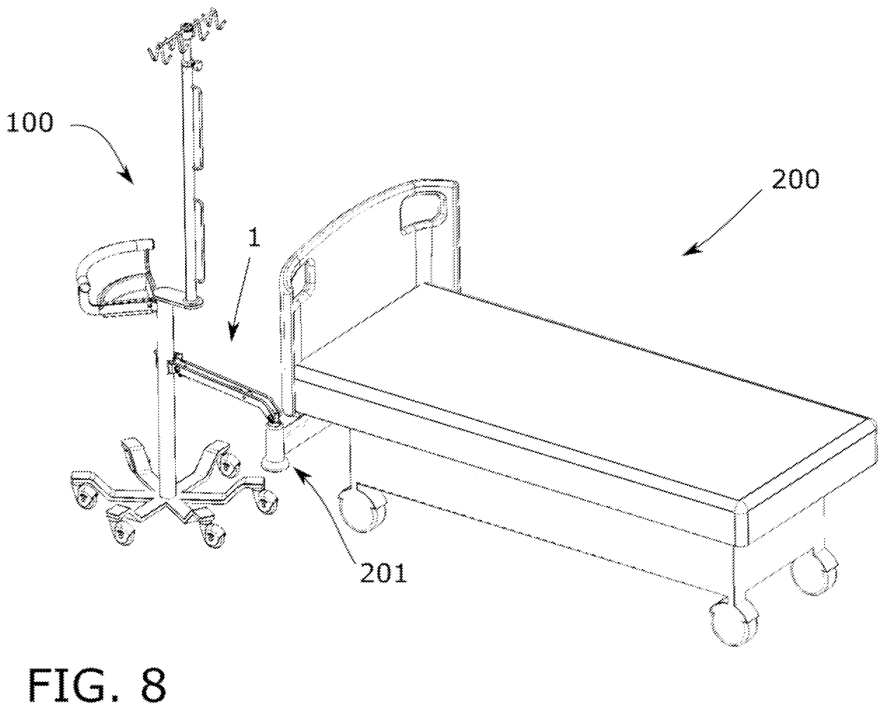

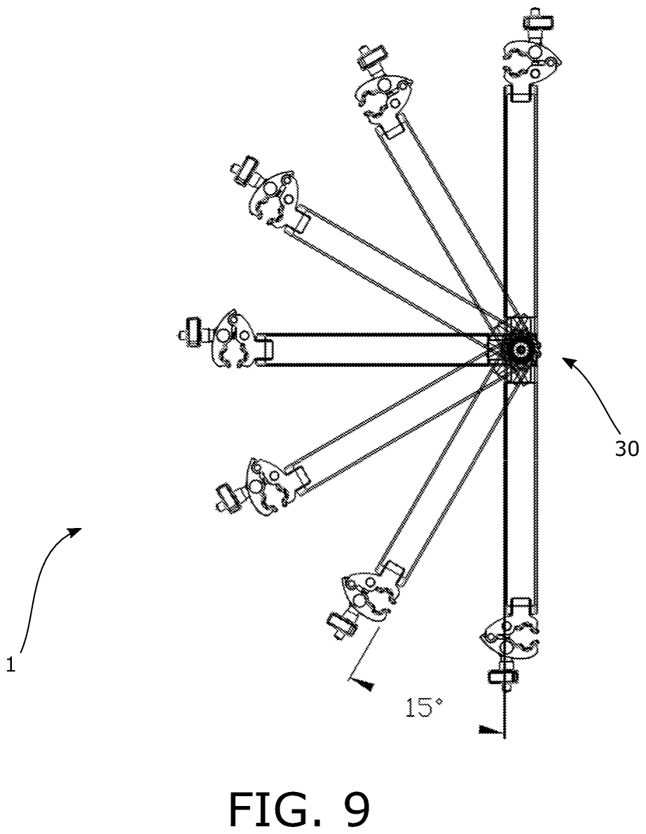

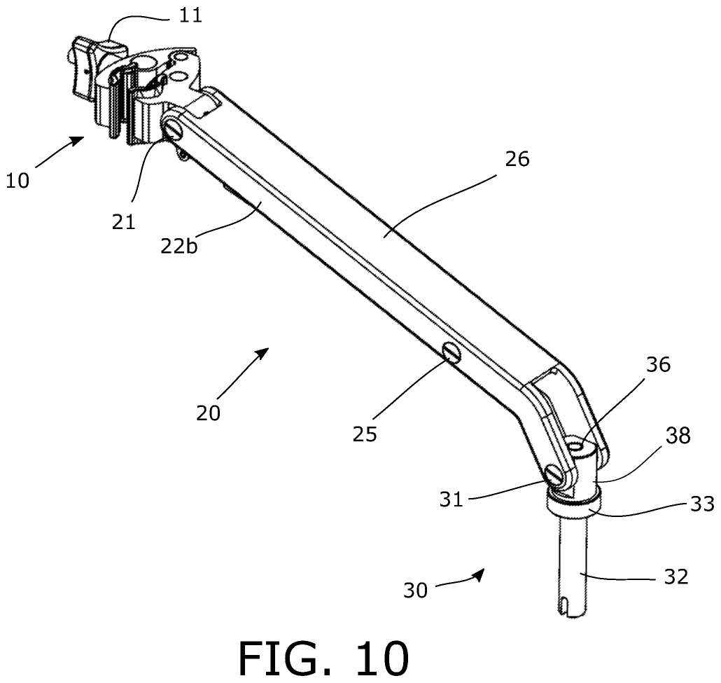

FIG. 8 shows an exemplary embodiment of the clamp system 1 with an exemplary clamped equipment 100 (e.g., a pole/stand for IV or other medical devices) and an exemplary support or frame (e.g., a bed) 200. The attachment pin 32 may be affixed to the receiving part 201 of the support 200 such that the clamp system 1 and clamped equipment 100 is configured to be adjustably positioned (e.g., rotate) about the longitudinal axis of the attachment pin 32. In certain embodiments, the attachment pin 32 may rotate relative to the receiving part 201. As shown in FIG. 9, the rotation of the link arm relative to the attachment pin may occur in 15 degree increments. Or, in the alternative, the receiving part 201 may rotate relative to the frame 200 while the connection between the pin 32 and the part 201 is fixed. The pin 32 and receiving part 201 can be configured as a male/female type connection. Alternatively, the pin 32 may be connected to the support and the receiving part 201 may be part of the clamp system 1. The clamp system 1 is configured to attach to the equipment 100 through the tightening and loosening of clamp portion 10 via the knob 11.

FIG. 9 shows an exemplary embodiment of the clamp system 1 adjustable moving with an exemplary adjustment angles of 15° increments. The clamp system 1 may also rotate in smaller or larger angle increments. The rotation of the link arm shown in FIG. 9 is useful, for example, for repositioning the IV pole when moving a bed an IV pole around a facility and into and out of elevators, doors, rooms, etc.

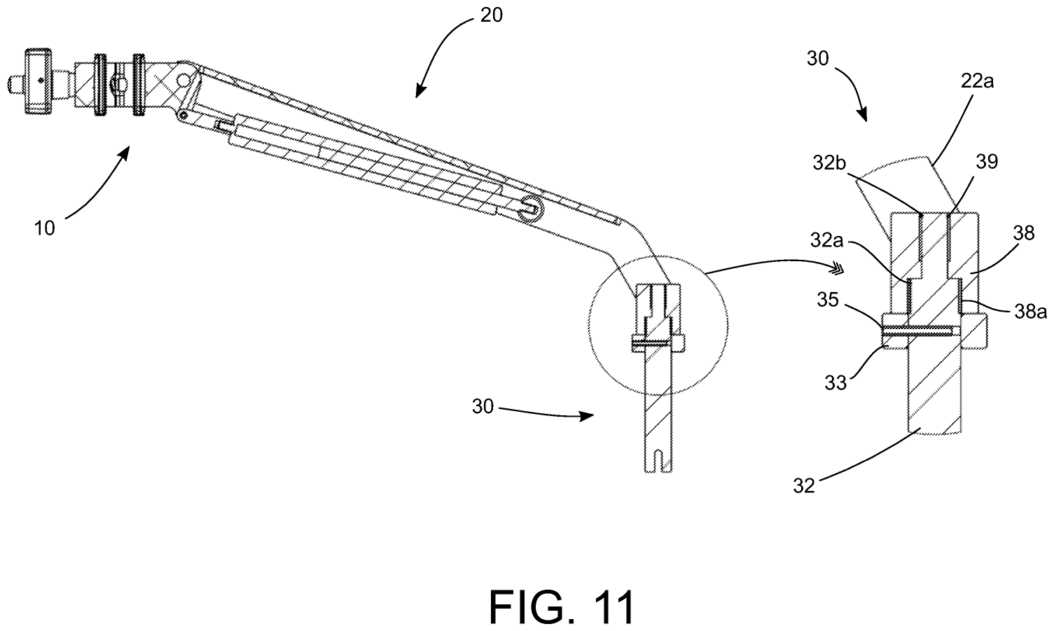

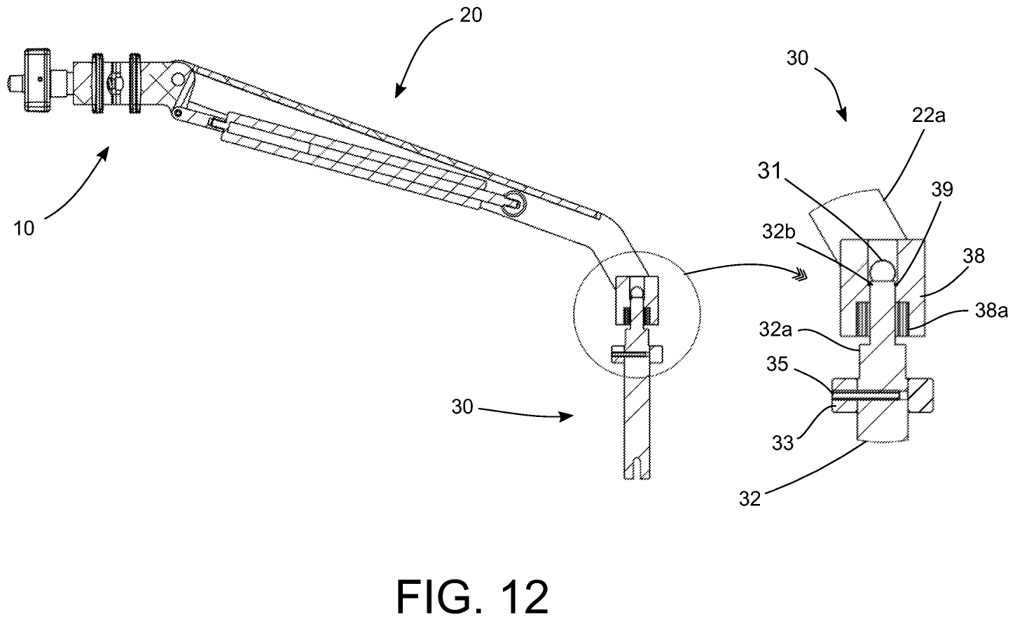

FIG. 10 shows an exemplary embodiment of clamp system 1 with a cover 26, covering a part of the link arm portion 20. In this embodiment, the attachment pin 32 may connect to a socket head 38 attached to link arms 22a and 22b. FIGS. 11 and 12 show sectional drawings of this embodiment. The socket head 38 may include a socket head chamfer interface 38a configured to engage a pin chamfer interface 32a on the attachment pin 32 which prevents the socket head 38 from rotating relative to the attachment pin 32 as shown in FIG. 11. The interface between surfaces 32a and 38a thus also prevents the link arm portion 20 and clamp 10 from rotating. The angles of the surfaces of the pin chamfer interface 32a and socket head chamfer interface 38a may correspond to the adjustment angles shown in FIG. 9. Any number of chamfered edges may be provided depending on the desired angle increments. The socket head 38 may be pulled upwards to disengage the socket head chamfer interface 38a from the pin chamfer interface 32a so that the socket head 38 may freely rotate as shown in FIG. 12. While in this disengaged state the link arm portion 20 and clamp 10 is allowed to freely rotate relative to the receiving part 201 of the support 200. A retaining ring 39 may be included to prevent the socket head 38 from being released completely from the attachment pin 32. The retaining ring 39 is configured to abut an interior surface of the socket head 38 when the socket head is completely pulled upwards in order to prevent the socket head 38 from being detached. The attachment ring 39 may be annular and surround a circumference of the attachment pin 32. The attachment pin 32 may include an annular recess 32b in order to hold the attachment ring 39 in place at the attachment ring 32. Alternatively, other suitable and conventional mechanisms may be used to provide an abutment or mechanical stop that prevents the socket head 38 from being completely separated from the attachment pin 32.

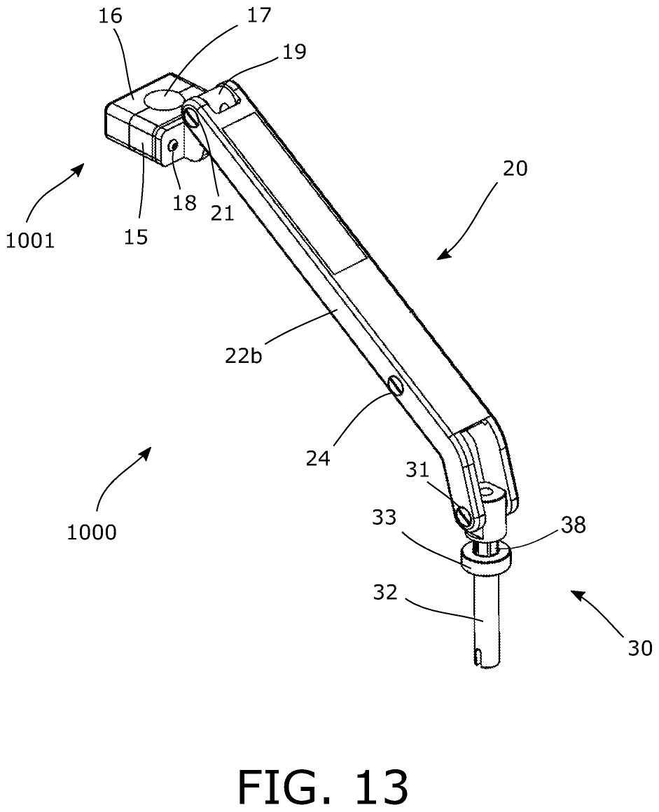

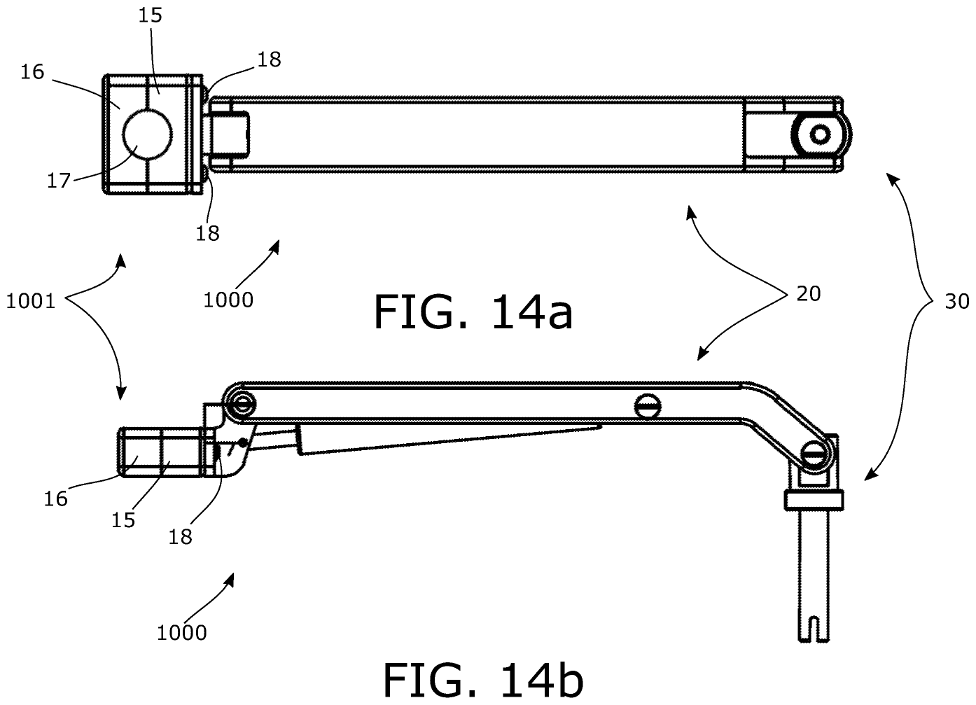

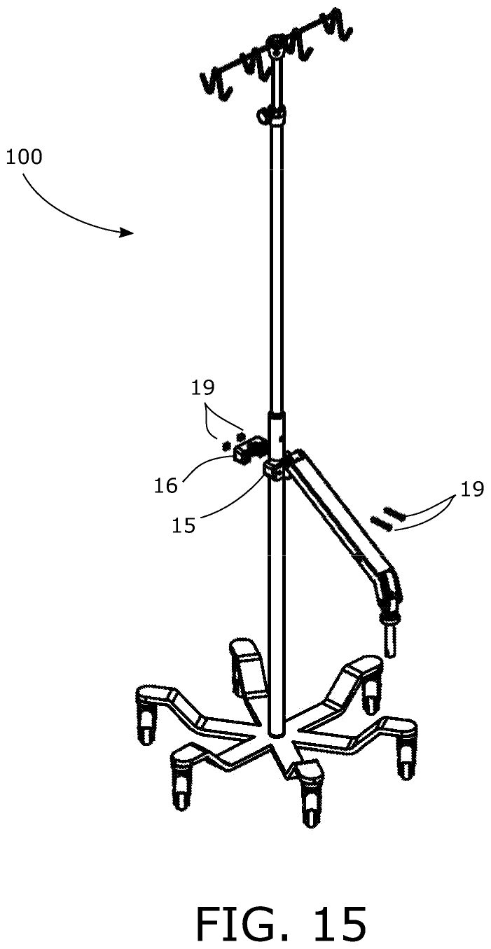

FIGS. 13, 14a, and 14b show an alternative exemplary embodiment of a clamp system 1000. In this embodiment, a clamp portion 1001 now includes a first mount block portion 15 and a connected second mount block portion 16. The first mount block 15 is configured to attach to the first end of the link arm portion 20 while the second mount block portion 16 is configured to be movable relative to and/or removable connected to the first mount block portion 15. A fastening mechanism (e.g. a nut and a bolt) is configured to be inserted into a fastening hole 18. The first mount block 15 may also include a hinge attachment configured to be connected to the arm link pivot 21 at the first end of the link arm portion 20. The clamp portion 1001 is configured to be rotatable around the arm link pivot 21. The first mount block portion 15 and second mount block portion 16 are separable in order to allow the clamp portion 1001 to be attached to equipment 100 (e.g., an IV pole). The equipment 100 is retained within the clamp opening 17 created by the two block portions 15 and 16. The fastening mechanism 19 may be inserted into the fastening hole 18 is configured to tighten and close the distance between the first mount block portion 15 and second mount block portion 16 in order to secure the clamp portion 1001 (i.e. hold on to by friction) to the equipment 100 as shown in FIG. 15. A plurality of fastening mechanisms and fastening holes may be provided.

Although described above as pins, bars or rods, the pivoting connections may include any combination of fasteners, bushings, bearings, nuts, bolts, and any form of suitable rotational joints.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

It is important to note that the construction and arrangement of the clamp system as shown in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

What is claimed is:

1. An attachment mechanism for pivotally connecting an upright beam to a support, the attachment mechanism comprising:
a clamp configured to hold the upright beam, wherein the clamp includes a first clamp arm, second clamp arm, and a knob attached to both first and second clamp arms;
a link arm pivotally connected to the clamp at a first end of the link arm;
an attachment portion connected to the link arm at a second end of the link arm, wherein the attachment portion is configured to be connected to the support;
wherein said knob is configured to adjust the distance between the first clamp arm and the second clamp arm such that the clamp holds the upright beam and prevents the upright beam from rotating relative to the clamp.

2. The attachment mechanism of claim 1, wherein the link arm comprises a first arm member and a second arm member.

3. The attachment mechanism of claim 2, wherein the link arm comprises a dampener located between the first arm member and the second arm member.

4. The attachment mechanism of claim 3, wherein the dampener is configured to be attached at the clamp at a first dampener end and the link arms at a second dampener end.

5. The attachment mechanism of claim 1, wherein the link arm and the dampener are configured to rotate relative to the clamp about a rotational axis perpendicular to the upright beam; and wherein the link arm is configured to adjustable rotate relative to the bed frame about an axis parallel to the upright beam.

6. The attachment mechanism of claim 1, further comprising a screw attached to the knob, wherein the screw is configured to extend through the first clamp arm.’

7. The attachment mechanism of claim 6, further comprising a bolt configured to fasten to an end of the screw, wherein the bolt is configured to extend through the second clamp arm.

8. An attachment mechanism for connecting an upright IV pole to a bed frame, the attachment mechanism comprising:
a clamp configured to hold the IV pole, wherein the clamp includes a first clamp arm, second clamp arm, and a knob attached to both first and second clamp arms;
a link arm connected to the clamp at a first end of the link arm;
an attachment portion connected to the link arm and the bed frame at a second end of the link arm, and wherein the link arm is configured to pivot relative to the bed frame about an axis of rotation that is perpendicular to the axis of the upright IV pole ;
wherein the knob is configured to rotate in order to adjust the distance between the first clamp arm and the second clamp arm such that the clamp holds the upright pole and prevents the upright pole from rotating relative to the clamp.

9. The attachment mechanism of claim 8, wherein the link arm comprises a first arm member and a second arm member.

10. The attachment mechanism of claim 9, wherein the link arm comprises a dampener located between the first arm member and the second arm member.

11. The attachment mechanism of claim 10, wherein the dampener is configured to be attached at the clamp at a first dampener end and the link arms at a second dampener end.

12. The attachment mechanism of claim 8, wherein the link arm and the dampener are configured to rotate relative to the clamp, about an axis of rotation that is perpendicular to the axis of the upright IV pole.

13. The attachment mechanism of claim 8, further comprising a screw attached to the knob, wherein the screw is configured to extend through the first clamp arm.

14. The attachment mechanism of claim 13, further comprising a bolt configured to fasten to an end of the screw, wherein the bolt is configured to extend through the second clamp arm.

15. An attachment mechanism comprising:
a clamp configured to hold a pole, wherein the clamp includes a first clamp arm, second clamp arm, and a knob attached to both first and second clamp arms;
a link arm connected to the clamp at a first end of the link arm, wherein the link arm is configured to pivot relative to the clamp about an axis that is perpendicular to an axis extending along the length of the pole;
an attachment portion connected to the link arm and a frame at a second end of the link arm, wherein the link arm is configured to pivot relative to the frame about an axis that is perpendicular to the axis of the pole;
wherein said knob is configured to adjust the distance between the first clamp arm and the second clamp arm such that the clamp holds the pole and prevents the pole from rotating relative to the clamp.

16. The attachment mechanism of claim 15, wherein the link arm comprises a first arm member and a second arm member.

17. The attachment mechanism of claim 16, wherein the link arm comprises a dampener located between the first arm member and the second arm member.

18. The attachment mechanism of claim 17, wherein the dampener is configured to be attached at the clamp at a first dampener end and the link arms at a second dampener end.

19. The attachment mechanism of claim 15, wherein the link arm and the dampener are configured to pivot relative to the clamp about a first pivot pin and wherein the link arm is configured to pivot relative to the frame about a second pivot pin.

20. The attachment mechanism of claim 15, further comprising a screw attached to the knob, wherein the screw is configured to extend through the first clamp arm.
